SEQUENCE LISTING (6 sequences)

SEQ ID NO: 1 - protein, 717 aa, Arabidopsis thaliana
MASSAFAFPSYIITKGGLSTDSCKSTSLSSSRSLVTDLPSPCLKPNNNSHSNRRAKVCASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 2 - protein, 659 aa, Arabidopsis thaliana
ASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 3 - protein, 824 aa, Artificial Sequence
  Residues 1-165 are from the thioredoxin sequence found in the vector pET32 supplied by Novagen. Residues 166-824 represent the tDXPS sequence from Arabidopsis shown in SEQ ID NO2.
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADIGSASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 4 - DNA, 2151 bp, Arabidopsis thaliana
atggcttcttctgcatttgcttttccttcttacataataaccaaaggaggactttcaactgattcttgtaaatcaacttctttgtcttcttctagatctttggttacagatcttccatcaccatgtctgaaacccaacaacaattcccattcaaacagaagagcaaaagtgtgtgcttcacttgcagagaagggtgaatattattcaaacagaccaccaactccattacttgacactattaactacccaatccacatgaaaaatctttctgtcaaggaactgaaacaactttctgatgagctgagatcagacgtgatctttaatgtgtcgaaaaccggtggacatttggggtcaagtcttggtgttgtggagcttactgtggctcttcattacattttcaatactccacaagacaagattctttgggatgttggtcatcagtcttatcctcataagattcttactgggagaagaggaaagatgcctacaatgaggcaaaccaatggtctctctggtttcaccaaacgaggagagagtgaacatgattgctttggtactggacacagctcaaccacaatatctgctggtttaggaatggcggtaggaagggatttgaaggggaagaacaacaatgtggttgctgtgattggtgatggtgcgatgacggcaggacaggcttatgaagccatgaacaacgccggatatctagactctgatatgattgtgattcttaatgacaacaagcaagtctcattacctacagctactttggatggaccaagtccacctgttggtgcattgagcagtgctcttagtcggttacagtctaacccggctctcagagagttgagagaagtcgcaaagggtatgacaaagcaaataggcggaccaatgcatcagttggcggctaaggtagatgagtatgctcgaggaatgataagcgggactggatcgtcactgtttgaagaactcggtctttactatattggtccagttgatgggcacaacatagatgatttggtagccattcttaaagaagttaagagtaccagaaccacaggacctgtacttattcatgtggtgacggagaaaggtcgtggttatccttacgcggagagagctgatgacaaataccatggtgttgtgaaatttgatccagcaacgggtagacagttcaaaactactaataagactcaatcttacacaacttactttgcggaggcattagtcgcagaagcagaggtagacaaagatgtggttgcgattcatgcagccatgggaggtggaaccgggttaaatctctttcaacgtcgcttcccaacaagatgtttcgatgtaggaatagcggaacaacacgcagttacttttgctgcgggtttagcctgtgaaggccttaaacccttctgtgcaatctattcgtctttcatgcagcgtgcttatgaccaggttgtccatgatgttgatttgcaaaaattaccggtgagatttgcaatggatagagctggactcgttggagctgatggtccgacacattgtggagctttcgatgtgacatttatggcttgtcttcctaacatgatagtgatggctccatcagatgaagcagatctctttaacatggttgcaactgctgttgcgattgatgatcgtccttcttgtttccgttaccctagaggtaacggtattggagttgcattacctcccggaaacaaaggtgttccaattgagattgggaaaggtagaattttaaaggaaggagagagagttgcgttgttgggttatggctcagcagttcagagctgtttaggagcggctgtaatgctcgaagaacgcggattaaacgtaactgtagcggatgcacggttttgcaagccattggaccgtgctctcattcgcagcttagctaagtcgcacgaggttctgatcacggttgaagaaggttccattggaggttttggctcgcacgttgttcagtttcttgctctcgatggtcttcttgatggcaaactcaagtggagaccaatggtactgcctgatcgatacattgatcacggtgcaccagctgatcaactagctgaagctggactcatgccatctcacatcgcagcaaccgcacttaacttaatcggtgcaccaagggaagctctgttt

SEQ ID NO: 5 - DNA, 1977 bp, Arabiodopsis thaliana
gcttcacttgcagagaagggtgaatattattcaaacagaccaccaactccattacttgacactattaactacccaatccacatgaaaaatctttctgtcaaggaactgaaacaactttctgatgagctgagatcagacgtgatctttaatgtgtcgaaaaccggtggacatttggggtcaagtcttggtgttgtggagcttactgtggctcttcattacattttcaatactccacaagacaagattctttgggatgttggtcatcagtcttatcctcataagattcttactgggagaagaggaaagatgcctacaatgaggcaaaccaatggtctctctggtttcaccaaacgaggagagagtgaacatgattgctttggtactggacacagctcaaccacaatatctgctggtttaggaatggcggtaggaagggatttgaaggggaagaacaacaatgtggttgctgtgattggtgatggtgcgatgacggcaggacaggcttatgaagccatgaacaacgccggatatctagactctgatatgattgtgattcttaatgacaacaagcaagtctcattacctacagctactttggatggaccaagtccacctgttggtgcattgagcagtgctcttagtcggttacagtctaacccggctctcagagagttgagagaagtcgcaaagggtatgacaaagcaaataggcggaccaatgcatcagttggcggctaaggtagatgagtatgctcgaggaatgataagcgggactggatcgtcactgtttgaagaactcggtctttactatattggtccagttgatgggcacaacatagatgatttggtagccattcttaaagaagttaagagtaccagaaccacaggacctgtacttattcatgtggtgacggagaaaggtcgtggttatccttacgcggagagagctgatgacaaataccatggtgttgtgaaatttgatccagcaacgggtagacagttcaaaactactaataagactcaatcttacacaacttactttgcggaggcattagtcgcagaagcagaggtagacaaagatgtggttgcgattcatgcagccatgggaggtggaaccgggttaaatctctttcaacgtcgcttcccaacaagatgtttcgatgtaggaatagcggaacaacacgcagttacttttgctgcgggtttagcctgtgaaggccttaaacccttctgtgcaatctattcgtctttcatgcagcgtgcttatgaccaggttgtccatgatgttgatttgcaaaaattaccggtgagatttgcaatggatagagctggactcgttggagctgatggtccgacacattgtggagctttcgatgtgacatttatggcttgtcttcctaacatgatagtgatggctccatcagatgaagcagatctctttaacatggttgcaactgctgttgcgattgatgatcgtccttcttgtttccgttaccctagaggtaacggtattggagttgcattacctcccggaaacaaaggtgttccaattgagattgggaaaggtagaattttaaaggaaggagagagagttgcgttgttgggttatggctcagcagttcagagctgtttaggagcggctgtaatgctcgaagaacgcggattaaacgtaactgtagcggatgcacggttttgcaagccattggaccgtgctctcattcgcagcttagctaagtcgcacgaggttctgatcacggttgaagaaggttccattggaggttttggctcgcacgttgttcagtttcttgctctcgatggtcttcttgatggcaaactcaagtggagaccaatggtactgcctgatcgatacattgatcacggtgcaccagctgatcaactagctgaagctggactcatgccatctcacatcgcagcaaccgcacttaacttaatcggtgcaccaagggaagctctgttt

SEQ ID NO: 6 - DNA, 2472 bp, Artificial Sequence
  Nucleotides 1-495 encode the thioredoxin sequence found in the vector pET32 supplied by Novagen. Nucleotides 496-2472 represent the tDXPS cDNA sequence from Arabidopsis thaliana.
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccgcttcacttgcagagaagggtgaatattattcaaacagaccaccaactccattacttgacactattaactacccaatccacatgaaaaatctttctgtcaaggaactgaaacaactttctgatgagctgagatcagacgtgatctttaatgtgtcgaaaaccggtggacatttggggtcaagtcttggtgttgtggagcttactgtggctcttcattacattttcaatactccacaagacaagattctttgggatgttggtcatcagtcttatcctcataagattcttactgggagaagaggaaagatgcctacaatgaggcaaaccaatggtctctctggtttcaccaaacgaggagagagtgaacatgattgctttggtactggacacagctcaaccacaatatctgctggtttaggaatggcggtaggaagggatttgaaggggaagaacaacaatgtggttgctgtgattggtgatggtgcgatgacggcaggacaggcttatgaagccatgaacaacgccggatatctagactctgatatgattgtgattcttaatgacaacaagcaagtctcattacctacagctactttggatggaccaagtccacctgttggtgcattgagcagtgctcttagtcggttacagtctaacccggctctcagagagttgagagaagtcgcaaagggtatgacaaagcaaataggcggaccaatgcatcagttggcggctaaggtagatgagtatgctcgaggaatgataagcgggactggatcgtcactgtttgaagaactcggtctttactatattggtccagttgatgggcacaacatagatgatttggtagccattcttaaagaagttaagagtaccagaaccacaggacctgtacttattcatgtggtgacggagaaaggtcgtggttatccttacgcggagagagctgatgacaaataccatggtgttgtgaaatttgatccagcaacgggtagacagttcaaaactactaataagactcaatcttacacaacttactttgcggaggcattagtcgcagaagcagaggtagacaaagatgtggttgcgattcatgcagccatgggaggtggaaccgggttaaatctctttcaacgtcgcttcccaacaagatgtttcgatgtaggaatagcggaacaacacgcagttacttttgctgcgggtttagcctgtgaaggccttaaacccttctgtgcaatctattcgtctttcatgcagcgtgcttatgaccaggttgtccatgatgttgatttgcaaaaattaccggtgagatttgcaatggatagagctggactcgttggagctgatggtccgacacattgtggagctttcgatgtgacatttatggcttgtcttcctaacatgatagtgatggctccatcagatgaagcagatctctttaacatggttgcaactgctgttgcgattgatgatcgtccttcttgtttccgttaccctagaggtaacggtattggagttgcattacctcccggaaacaaaggtgttccaattgagattgggaaaggtagaattttaaaggaaggagagagagttgcgttgttgggttatggctcagcagttcagagctgtttaggagcggctgtaatgctcgaagaacgcggattaaacgtaactgtagcggatgcacggttttgcaagccattggaccgtgctctcattcgcagcttagctaagtcgcacgaggttctgatcacggttgaagaaggttccattggaggttttggctcgcacgttgttcagtttcttgctctcgatggtcttcttgatggcaaactcaagtggagaccaatggtactgcctgatcgatacattgatcacggtgcaccagctgatcaactagctgaagctggactcatgccatctcacatcgcagcaaccgcacttaacttaatcggtgcaccaagggaagctctgttt

(19) United States
(12) Patent Application Publication
(10) Pub. No.: US 2002/0168743 A1
(43) Pub. Date: Nov. 14, 2002

(54) Methods and compositions for the identification of modulators of deoxyxylulose 5-phosphate synthase activity

(76) Inventors: John W. Rice, Pittsboro, NC (US); Andreas S. Kloti, Richterswil, NC (US); John M. Crawford JR., Raleigh, NC (US); Beth Lanning, Cary, NC (US); Sandy J. Stewart, Durham, NC (US)

Correspondence Address: PARADIGM GENETICS, INC, 108 ALEXANDER DRIVE, P O BOX 14528, RTP, NC, 27709-4528, US

(21) Appl. No.: 10/046,583

(22) Filed: Oct. 26, 2001

Related U.S. Application Data
(62) Division of application No. 09/626,589, filed on Jul. 27, 2000, now Pat. No. 6,326,164.

(51) Int. Cl. C12Q 1/26; C12Q 1/42; C07H 21/04; C12N 9/16; C12P 21/02; C12N 5/04; C12N 1/21

(52) U.S. Cl. 435/196; 435/21; 435/69.1; 435/252.3; 435/419; 435/320.1; 536/23.2

(57) ABSTRACT

The invention is directed to methods and compositions for the determination of deoxyxylulose 5-phosphate synthase (DXPS) activity. The methods and compositions of the invention are amenable to high throughput screening assays for the identification of inhibitors and enhancers of DXPS activity. Such compounds have use in the modulation of plant and microbial growth. The compositions of the invention are DXPS fragments and chimeric polypeptides that have increased solubility as compared to the wild type DXPS polypeptide. These DXPS fragments and chimeric polypeptides, or variants thereof, can be recombinantly expressed and purified in quantities suitable for high throughput screening assays. The assays of the invention are based on the detection of substrates of DXPS that remain after a DXPS reaction.

### CROSS-REFERENCE TO RELATED APPLICATION

This Application is a Divisional of U.S. application Ser. No. 09/626,589 Filed Jul. 27, 2000, herein incorporated by reference in its entirety.

### FIELD OF THE INVENTION

The present invention relates to assays for measuring deoxyxylulose 5-phosphate synthase (DXPS) activity. The assays can be used to identify compounds that inhibit or enhance DXPS activity. Such compounds have use in modulating plant and microbial growth and development.

### BACKGROUND OF THE INVENTION

Deoxy-D-xylulose 5-phosphate (DXP) is a common precursor of thiamin (vitamin B1), pyridoxyl (vitamin B6) and isoprenoids. Isoprenoids encompass a large family of biomolecules, including vitamins A, D, E and K, cholesterol, plant pigments such as carotenoids and the phytol chain of chlorophyll, natural rubber, many essential oils, plant hormones (gibererellins, abscisic acid), insect juvenile hormone, dolichols, quinone electron carriers in mitochondria and chloroplasts, such as ubiquinone and plastoquinone, structural components of membranes (phytosterols) and Ras protein. In higher plants and bacteria, the first step in the formation of isopentenyl diphosphate, the common precursor of all isoprenoids, by the mevalonate-independent pathway is the formation of DXP from the precursors pyruvate and glyceraldehyde 3-phosphate (FIG. 1). The reaction is catalyzed by the enzyme deoxyxylulose 5-phosphate synthase (DXPS) (Lange et al. (1998) Proc Natl Acad Sci 95:2100-2104; Lois et. al. (1998) Proc Natl Acad Sci 95:2105-2110; Sprenger et al. (1997) Proc Natl Acad Sci 94:12857-12862).

The DXPS genes or cDNAs from E. coli (GenBank AF035440), Hemophilus influenzae (Swiss-Prot P54205), Rhodobacter capsulatus (Swiss-Prot P26242), Synechocystus sp. PCC6803 (GenBank D90903), Bacillus subtilis (Swiss-Prot P54523), Helicobacter pylori (GenBank AE000552), Mycoplasma tuberculosis (GenBank Z96072), Glycine max (GenBank AW278762), Lycopersicon esculentum (GenBank AF143812), Catharanthus roseus (GenBank AJ0111840), Mentha x peperita (GenBank AF019383) and Arabidopsis thaliana (GenBank AF010383 and 5281015)have been cloned. Also, ESTs encoding fragments of DXPS have been identified in Oryza sativa, Ricinus communis, and Pinus taeda. However, no homologues of the DXPS genes have been identified in animals.

Disruption of the DXPS gene in Arabidopsis results in an albino phenotype due to a lack of chlorophyll and carotenoid pigments. These results indicate that DXPS is essential for chloroplast function (Lange et al.) and that inhibitors of DXPS activity may have use as herbicides. Accordingly, it would be useful to have a DXPS assay that is amenable to high throughput screening of herbicide candidates.

Several assays for DXPS activity have been reported in the literature. These assays are based on detection of the product, DXP. In these assays, the conversion of [2-¹⁴C]-pyruvate to [¹⁴C]-DXP in the presence of glyceraldehyde 3-phosphate and DXPS was measured by detecting [¹⁴C]-DXP using either reverse phase HPLC (Lange, et. al.) or thin layer chromatography (Lois et al.). However, neither format is suitable for high throughput screening.

### SUMMARY OF THE INVENTION

The invention is directed to methods and compositions for the determination of deoxyxylulose 5-phosphate synthase (DXPS) activity. The methods and compositions of the invention are amenable to high throughput screening assays for the identification of inhibitors and enhancers of DXPS activity. Such compounds have use in the modulation of plant growth and development.

The compositions of the invention are DXPS fragments and chimeric polypeptides that have increased solubility in cell extracts as compared to the wild type DXPS polypeptide. These DXPS fragments and chimeric polypeptides can be recombinantly expressed and purified in quantities suitable for high throughput screening assays.

The assays of the invention are based on the detection of substrates of DXPS that remain after a DXPS reaction. Specifically, the invention provides a method for determining deoxyxylulose 5-phosphate synthase activity, comprising:

a) contacting pyruvate and optionally, glyceraldehyde 3-phosphate, with a deoxyxylulose 5-phosphate synthase; and

b) determining the concentration of pyruvate and/or glyceraldehyde 3-phosphate remaining after the contact in step (a).

The assays of the invention are useful for the identification of modulators of deoxyxylulose 5-phosphate synthase activity. Thus, in another aspect, the invention provides a method for identifying modulators of deoxyxylulose 5-phosphate synthase activity, comprising:

a) contacting pyruvate and optionally, glyceraldehyde 3-phosphate, with a deoxyxylulose 5-phosphate synthase, in the presence and the absence of at least one candidate modulator; and

b) comparing the concentration of pyruvate and/or glyceraldehyde 3-phosphate remaining after said contact in the absence of said candidate modulator to said concentration in the presence of said candidate modulator.

### BRIEF DESCRIPTION OF THE DRAWINGS

FIG. 1. Schematic diagram showing the conversion of pyruvate and glyceraldehyde 3-phosphate (G 3-P) to deoxyxylulose 5-phosphate (DXP) by deoxyxylulose 5-phosphate synthase (DXPS).

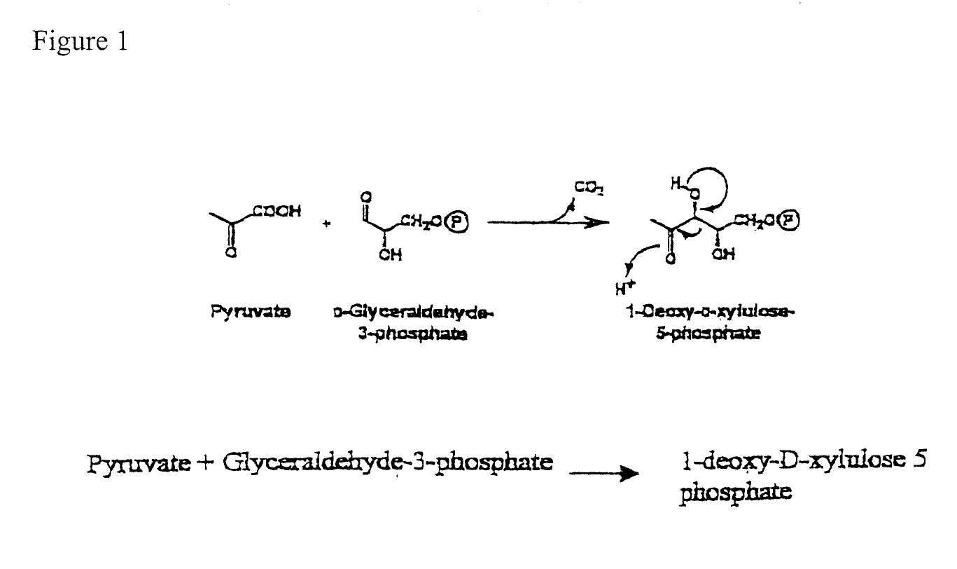

FIG. 2. Schematic diagram of the trxA/tDXPS chimeric polypeptide.

FIG. 3. Coomassie stained SDS-page gel of tDXPS purification. Lane 1) Molecular weight markers as indicated, lane 2) Clarified E. coli supernate, lane 3) Resuspended insoluble pellet, lane 4) Column flow-through, lane 5) Purified tDXPS.

FIG. 4. Effect of DXPS enzyme on pyruvate concentration as determined by the conversion of NADH to NAD in the presence of pyruvate and lactate dehydrogenase (LDH).

FIG. 5. Rate of conversion of pyruvate and NADH to lactic acid and NAD by lactate dehydrogenase.

FIG. 6. Standard curve of NADH concentration using 340 nm excitation/460 nm emission fluorescence of NADH.

FIG. 7. Standard curve of pyruvate concentration by fluorescence of NADH following a lactate dehydrogenase reaction. The relative fluorescence of NADH at 340 nm excitation-460 nm emission is shown.

FIG. 8. Determination of reaction time for tDXPS as measured by NADH fluorescence in a lactate dehydrogenase reaction. ♦-DXPS, ▪-E. coli crude extract.

FIG. 9. Total Activity at a 3-hour time point for various amounts of tDXPS protein. Values are the mean of triplicate determinations, with standard deviation indicated. 1 μg/well of protein was chosen for all further experiments.

FIG. 10. Time course of DXPS reaction in the presence and absence of glyceraldehyde 3-phosphate (g-3-p).

### DETAILED DESCRIPTION

The present invention discloses methods and compositions for the measurement of the activity of the enzyme deoxyxylulose 5-phosphate synthase (DXPS). In contrast to prior art assays, the assays of the invention are amenable to high throughput screening protocols. Such assays are useful in the rapid identification of inhibitors and enhancers of DXPS activity. Inhibitors of DXPS activity have use as herbicides and as antimicrobial agents. Enhancers of DXPS activity can be used to modulate vitamin B1, vitamin B2 and isoprenoid production in plants and microorganisms.

The compositions of the invention comprise soluble derivatives of Arabidopsis thaliana DXPS protein. The full length Arabidopsis thaliana DXPS cDNA has previously been reported and is shown in SEQ ID NO:1. However, expression of the full length DXPS protein in baculovirus or E. coli expression systems failed to yield soluble protein.

A putative 66 amino acid chloroplast targeting sequence for A. thaltiana DXPS has been reported in the literature. Sprenger et al. (1997) Proc Natl Acad Sci 94:12857-12862. In contrast, we predicted that the targeting sequence corresponded to the N-terminal 58 amino acids of the DXPS protein. The sequence of the truncated A. thaliana DXPS protein (tDXPS), from which the N-terminal 58 amino acids have been removed, is shown in SEQ ID NO:2. As discussed below, this truncated protein possesses DXPS activity.

Thus, in one aspect, the invention provides a polypeptide consisting essentially of SEQ ID NO:2. For the purposes of the invention, a polypeptide consisting essentially of SEQ ID NO:2 is limited to the polypeptide of SEQ ID NO:2 and optionally, one to seven additional amino acid residues on the amino and/or carboxy terminus of SEQ ID NO:2.

By “polypeptide” is meant a chain of at least four amino acids joined by peptide bonds. The chain may be linear, branched, circular or combinations thereof The polypeptides may contain amino acid analogs and other modifications, including, but not limited to glycosylated or phosphorylated residues.

In another aspect, the invention provides a polynucleotide consisting essentially of a nucleic acid encoding the polypeptide of SEQ ID NO:2. In addition, the invention provides an expression cassette comprising an isolated polynucleotide consisting of a nucleic acid encoding the polypeptide of SEQ ID NO:2.

For the purposes of the invention, an “isolated polynucleotide” is a polynucleotide that is substantially free of the nucleic acid sequences that normally flank the polynucleotide in its naturally occurring replicon. For example, a cloned polynucleotide is considered isolated. Alternatively, a polynucleotide is considered isolated if it has been altered by human intervention, or placed in a locus or location that is not its natural site, or if it is introduced into cell by agroinfection.

As used herein, “nucleic acid” and “polynucleotide” refers to RNA or DNA that is linear or branched, single or double stranded, or a hybrid thereof The term also encompasses RNA/DNA hybrids. Less common bases, such as inosine, 5-methylcytosine, 6-methyladenine, hypoxanthine and others can also be used. Other modifications, such as modifications to the phosphodiester backbone, or the 2-hydroxy in the ribose sugar group of the RNA can also be made.

The polynucleotides of the invention can be inserted into expression cassettes and expression vectors for the production of recombinant DXPS protein. A variety of expression cassettes and vectors are known to those skilled in the art. The expression cassettes of the invention contain 5′ and 3′ regulatory sequences necessary for transcription and termination of the polynucleotide of interest. Thus, the expression cassettes will include a promoter and a transcriptional terminator. Other functional sequences may be included in the expression cassettes of the invention. Such functional sequences include, but are not limited to, introns, enhancers and translational initiation and termination sites and polyadenylation sites. The control sequences can be those that can function in at least one microorganism, insect cell or plant cell. These sequences may be derived from one or more genes, or can be created using recombinant technology.

Promoters useful in the expression cassettes of the invention include any promoter that is capable of initiating transcription in a microorganism, insect cell or plant cell. The promoter may be constitutive, inducible or tissue-preferred.

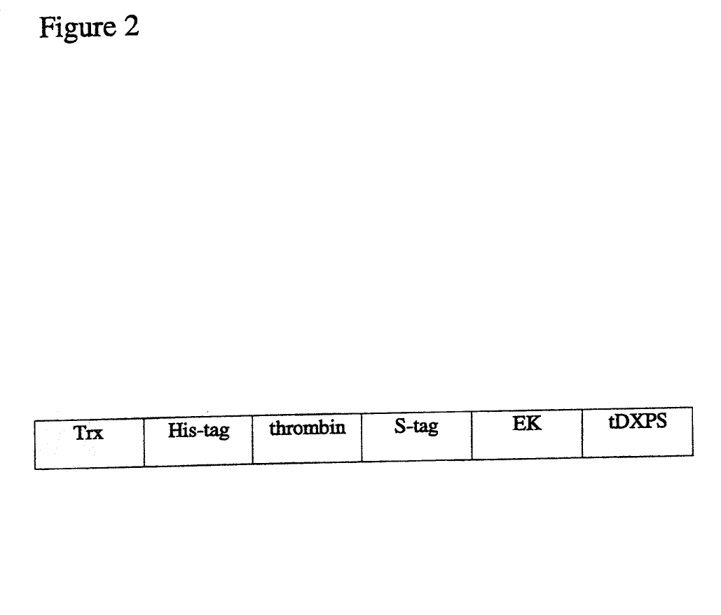

Expression in E. Coli of TDXPS as a thioredoxin fusion protein (trxA/fDXPS), the sequence of which is shown in SEQ ID NO:3 and diagrammed in FIG. 2, yielded quantities of soluble active protein sufficient for the development of a high throughput screening assay for DXPS activity. Thus, the invention provides a polypeptide comprising SEQ ID NO:3. In addition, the invention provides an isolated polynucleotide comprising a nucleic acid encoding the polypeptide of SEQ ID NO:3.

In another aspect, the invention provides assays for DXPS activity. DXPS catalyzes the conversion of pyruvate and glyceraldehyde 3-phosphate (G-3-P) to 1-deoxyxylulose 5-phosphate (DXP). Prior art assays for DXPS activity have measured the amount of [C¹⁴]-DXP produced in a reaction using [C¹⁴]-labeled substrate. DXP concentration was then determined by HPLC or TLC analysis [C¹⁴]-DXP. Such assays are not suitable for high throughput screening assays for DXPS activity.

In contrast to the prior art assays, the invention provides assays for DXPS activity based on a determination of the amount of substrate (pyruvate and/or G-3-P) remaining after a DXPS reaction. Surprisingly, we found that DXPS reacts with pyruvate in the absence of glyceraldehyde 3-phosphate. While no DXP is produced in this reaction, the concentration of pyruvate is depleted.

Thus, in one aspect, the invention provides a method for determining DXPS activity, comprising:

a) contacting pyruvate and optionally, glyceraldehyde 3-phosphate, with a deoxyxylulose 5-phosphate synthase; and

b) determining the concentration of pyruvate and/or glyceraldehyde 3-phosphate remaining after the contact in step (a).

The concentration of pyruvate and/or glyceraldehyde 3-phosphate remaining after this contact is inversely related to DXPS activity.

By deoxyxylulose 5-phosphate synthase (DXPS) is meant any enzyme that catalyzes the conversion of pyruvate and glyceraldehyde 3-phosphate to deoxyxylulose 5-phosphate. The DXPS may be a naturally occuring DXPS enzyme from any organism, an enzymatically active fragment of a naturally occuring DXPS enzyme, or a variant of a naturally occurring DXPS enzyme. Preferably, the DXPS is a plant DXPS or a prokaryotic DXPS. By plant DXPS is meant any DXPS enzyme that naturally occurs in at least one plant. Preferred plant DXPS enzymes include Arabidopsis thaliana DXPS, tDXPS (SEQ ID NO:2) and trxA/tDXPS (SEQ ID NO:3). By procaryotic DXPS is meant any DXPS enzyme that naturally occurs in at least one procaryote. Preferred procaryotic DXPS enzymes are from Hemophilus influenzae, Rhodobacter capsulatus, Synechocystus sp. PCC683, Bacillus subtilis, Helicobacter pylori and Mycoplasma tuberculosis.

As used herein, “enzymatically active fragments of a naturally occuring DXPS” refer to a polypeptide comprising at least 30 consecutive amino acids of the naturally occuring DXPS polypeptide and capable of catalyzing the conversion of pyruvate and glyceraldehyde 3-phosphate to DXP with at least 10% or more of the efficiency of the Arabidopsis tDXPS polypeptide represented as SEQ ID NO:2. The catalytic activity of any DXPS enzyme, fragment or variant thereof can be determined according to the method described in Example 5 below.

As used herein, “variant of a naturally occurring DXPS enzyme” refers to a polypeptide having at least 80% amino acid similarity with a naturally occuring DXPS polypeptide and capable of catalyzing the conversion of pyruvate and glyceraldehyde 3-phosphate to DXP with at least 10% or more of the efficiency of the Arabidopsis tDXPS polypeptide represented as SEQ ID NO:2.

Amino acid sequence similarity refers to amino acid residue positions in polypeptides that differ by conservative amino acid substitutions. An amino acid substitution is conservative if the substituted amino acid residue has similar chemical properties (e.g. charge or hydrophobicity) to the reference amino-acid residue and therefore does not substantially change the functional properties of the polypeptide. In general, a substitution of an amino acid for another amino acid having the same type of R group is considered a conservative substitution. Amino acids can be classified into the following R groups: nonpolar, aliphatic; polar, uncharged; positively charged; negatively charged; and aromatic. Glycine, alanine, valine, leucine, isoleucine and proline have nonpolar aliphatic R groups. Serine, threonine, cysteine, methionine, asparagine and glutamine have polar uncharged R groups. Lysine, arginine and histidine have positively charged R groups. Aspartate and glutamate have negatively charged R groups. Phenylalanine, tyrosine and tryptophan have aromatic R groups.

The percent similarity between amino acid sequences can be determined using the “FASTA” similarity search algorithm of Pearson and Lipman (Proc Natl Acad Sci USA 85:2444, 1988) and Pearson (Meth Enzymol 183:63, 1990). Illustrative parameters for FASTA analysis are: ktup=1, gap opening penalty=10, gap extension penalty=1, and substitution matrix=1BLOSUM62. These parameters can be introduced into a FASTA program by modifying the scoring matrix file (“SMATRIX”), as explained in Appendix 2 of Pearson, 1990 (ibid.).

In the DXPS assays of the invention, pyruvate and optionally, glyceraldehyde 3-phosphate (G-3-P) are contacted with DXPS enzyme. Typically, the contact of pyruvate and G-3-P with DXPS will be made by combining these compounds in an aqueous solution that is compatible with DXPS activity.

Optimal buffer conditions, reagent concentrations, times and temperatures for the DXPS reaction can be determined by one skilled in the art. Preferably, the aqueous solution comprises 10-100 mM Tris pH 7.5; 5-100 μM pyruvate; 0-100 μM NADH; 1-50 mM DTT; 0.8-25 mM MgCl₂; and 0.08-5 mM ThDp. Most preferably, the aqueous solution comprises 50 mM Tris pH 7.5; 30 μM pyruvate; 25 μM NADH; 5 mM DTT; 2.5 mM MgCl₂; and 0.3 mM ThDp. If G-3-P is present, preferably the concentration is 5-200 μM and most preferably about 25 μM. The amount of DXPS protein will depend on the purity and activity of the DXPS preparation. For Arabidopsis tDXPS prepared as described in Example 2, the preferred amount is 500-1000 ng/50 μl reaction. Preferably, the DXPS reaction is conducted at approximately 37° C. and allowed to proceed for 30 minutes to three hours.

Following the contact of DXPS with pyruvate and optionally, G-3-P, the concentration of one or more DXPS substrate remaining is determined. It will be understood that the DXPS reaction need not proceed to completion prior to determining the concentration of the remaining pyruvate or glyceraldehyde 3-phosphate.

In a preferred embodiment, the concentration of pyruvate remaining after the contact with DXPS is determined. Methods for measuring the concentration of pyruvate are known to those skilled in the art. For example, the concentration of pyruvate can be determined by HPLC, by a pyruvate kinase assay or through the use of the pyruvate diagnostic kit such as the one provided by Sigma. By HPLC is meant high performance liquid chromatography.

In addition, pyruvate is a substrate for other reactions. Most notable is the conversion of pyruvate by lactate dehydrogenase (LDH; E.C. 1.1. 1.27) in the presence of NADH to yield lactate and NAD. In a preferred embodiment, the concentration of pyruvate is determined by contacting the remaining pyruvate with lactate dehydrogenase and NADH and then determining the concentration of NADH. By NADH is meant β-nicotinamide adenine dinucleotide, reduced form. By NAD is meant β-nicotinamide adenine dinucleotide. The structures of NAD and NADH are described in Lehninger et al. Principles of Biochemistry, 2^{nd} Ed. Worth Publishers, New York, 1993.

Typically, the contact of pyruvate with NADH and LDH will be made by combining these compounds in an aqueous solution that is compatible with LDH activity. Optimal buffer conditions, reagent concentrations, times and temperatures for the LDH reaction can be determined by one skilled in the art. Preferably, the aqueous solution comprises 10-100 mM Tris pH 7.5; 1-100 μM NADH; 1-100 μM pyruvate and 0.2-10 units/ml LDH. Most preferably, the aqueous solution comprises 50 mM Tris pH 7.5, 25 μM NADH; 30 μM pyruvate and 2.5 units/ml LDH. Preferably, the LDH reaction is conducted at approximately room temperature and allowed to proceed for 1-10 minutes.

Following the contact of pyruvate and NADH with LDH, the concentration of NADH remaining can be determined. It will be understood that the LDH reaction need not proceed to completion prior to determining the concentration of the remaining NADH.

Methods for determining NADH concentration are known to those skilled in the art. Such methods include measurements of fluorescence and optical absorption. In one method, the concentration of NADH is determined by measuring the absorbance of NADH at approximately 320-360 nm, and preferably, at approximately 340 nm. More preferably, the concentration of NADH is determined by measuring the fluorescence of NADH at 340 nm excitation/460 nm emission.

As an alternative to determining the concentration of NADH, the concentration of NAD produced by contacting pyruvate with lactate dehydrogenase and NADH can be determined by measuring the absorbance of NAD at approximately 250-270 nm, and preferably at approximately 260 nm.

As an alternative to determining the concentration of pyruvate remaining after a DXPS reaction, the concentration of G-3-P remaining after this reaction can be determined. G-3-P can be measured by methods known to those skilled in the art, such as HPLC. In addition, G-3-P, like pyruvate, is a substrate for other reactions. For example, glyceraldehyde 3-phosphate and NAD are converted to 3-phosphoglycerate and NADH by glyceraldehyde 3-phosphate dehydrogenase (GAPH). Accordingly, following a glyceraldehyde 3-phosphate dehydrogenase reaction, the amount of NADH formed could be determined according to the methods described above.

The methods of the invention are particularly useful for identifying compounds that modulate DXPS activity. Such compounds are useful for the regulation of plant growth and development. For example, compounds that inhibit plant DXPS activity can be used as herbicides. Compounds that enhance DXPS activity can be used to increase production of thiamin (vitamin B1), pyridoxyl (vitamin B6) and isoprenoids in plants and other organisms.

Thus, the invention provides a method for identifying modulators of DXPS activity, comprising:

a) contacting pyruvate and optionally, glyceraldehyde 3-phosphate, with a deoxyxylulose 5-phosphate synthase, in the presence and the absence of at least one candidate compound; and

b) comparing the concentration of pyruvate and/or glyceraldehyde 3-phosphate remaining after said contact in the absence of said candidate compound to said concentration in the presence of said candidate compound.

An increase in the concentration of pyruvate or glyceraldehyde 3-phosphate in the presence of the candidate compound would indicate that the candidate compound is an inhibitor of DXPS activity. A decrease in the concentration of pyruvate or glyceraldehyde 3-phosphate in the presence of the candidate compound would indicate that the candidate compound is an enhancer of DXPS activity.

### Experimental

### EXAMPLE 1

### Cloning of A. thaliana DXPS cDNA and Expression in E. coli

The full-length cDNA for DXPS from A. thaliana was cloned using RT-PCR and inserted into a variety of expression vectors. Expression of the full length DXPS protein using baculovirus and E. coli expression systems failed to yield soluble protein. Similarly, expression of DXPS in E. coli as chimeric fusion proteins utilizing either N-terminal or C-terminal HIS-tag fusions or a thioredoxin fusion resulted in the association of recombinant DXPS with the insoluble fraction of the cell.

Our analysis of the full-length DXPS cDNA suggested that the first 58 amino acids encoded by this cDNA correspond to a plastid targeting sequence. In contrast, the prior art has predicted a 66 amino acid targeting sequence for Arabidopsis DXPS (Sprenger et al. (1997) Proc Natl Acad Sci 94:12857-12862). Utilizing RT-PCR on total RNA isolated from 14 day old Arabidopsis thaliana seedlings, we obtained a cDNA sequence for a truncated version of DXPS with the putative 58 amino acid targeting sequence removed (tDXPS). The truncated cDNA encoding tDXPS was ligated into the E. coli. expression vector pET32 (Novagen, Inc.). This expression vector allows for the expression of recombinant protein as a fusion product with thioredoxin (trxA). The expression vector also contains both a S-tag and a HIS sequence for purification by affinity or nickel chromatography, respectively, and both a thrombin protease cleavage site and an enterokinase (EK) protease cleavage site for removal of the Trx portion of the fusion protein (FIG. 2). Expression of the thioredoxin/tDXPS fusion protein in E. coli yielded quantities of soluble, active protein sufficient for the development of a high throughput screening (HTS) assay.

### EXAMPLE 2

### Purification of tDXPS

pET32/tDXPS was transformed into E. coli AD494(DE3)lysS (Novagen), following the manufacturer's instructions. Transformed bacteria were grown in LB liquid media at 37° C. to an optical density of ˜0.6 at 600 nm. At that point, isopropylthio-beta-galactoside (IPTG) was added to a final concentration of 1 mM and the culture was incubated at 37° C. for 4 additional hours. Bacteria were pelleted via centrifugation.

An E. coli pellet from 500 ml of an induced culture was lysed using BugBuster Bacteria Lysis Solution (Novagen) following the recommended protocol with the following modification. 20 μl of benzonase was used in the lysis step to help remove the DNA quickly from the cell lysate. This resulted in more complete lysis and reduced the viscosity of the mixture. The cell lysate was then clarified by centrifugation at 15,000×g for 10 minutes.

A volume of N₁-agarose beads sufficient to form a 5 ml column bed volume was equilibrated by washing twice with a 5×volume of Column Buffer (50 mM Tris, pH 7.5, 150 mM NaCl, 2.5 mM MgCl₂, 1 mM thiamin diphosphate (ThDp), 1 mM 2-mercaptoethanol). The supernate from the centrifuiged cell lysate was then added to the equilibrated Ni-agarose beads. The supernate/Ni-agarose mixture was incubated on ice for approximately 20 minutes, with occasional mixing to keep the beads in suspension.

The supernate/Ni-agarose mixture was then poured into a column and the supernate was allowed to flow through. The column was washed with 50 ml of Wash Buffer (50 mM Tris, pH 7.5, 300 mM NaCl, 2.5 mM MgCl₂, 1 mM ThDp, 1 mM 2-mercaptoethanol, 20 mM imidazol).

Bound protein was eluted with Elution Buffer (50 mM Tris, pH 7.5, 150 mM NaCl, 500 mM imidazol, 2.5 mM MgCl₂, 1 mM ThDp, 1 mM 2-mercaptoethanol). Fractions containing protein as determined by a Bio-Rad™ protein assay were pooled, and concentrated to ˜50% of the original volume using a 30,000 molecular weight cutoff spin filter.

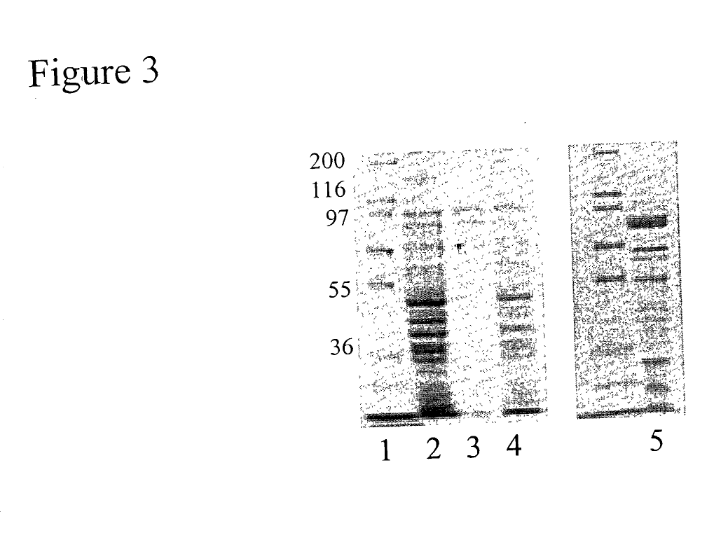

Because of potential imidazole interference in detection of NADH fluorescence, pooled protein was then dialyzed (1:500) against Dialysis Buffer (50 mM Tris, pH 7.5, 150 mM NaCl, 2.5 mM MgCl₂, 1 mM ThDp, 5 mM DTT) twice, for 1 hour each time, at 4° C. A Pierce “Slide-a-lyser” cassette with a 10,000 molecular weight cutoff membrane was used for the dialysis. Purification was monitored by SDS-PAGE (FIG. 3). Typically, tDXPS is the major protein band comprising ˜50% of the total protein in the purified sample.

The final protein concentration was determined using a BioRad protein assay kit. The protein was then flash-frozen and stored at −80° C. From a 500 ml E. coli culture (˜1.1 grams) we routinely obtain 2-2.5 mg of purified protein, or −0.2% of the total cell pellet weight. When this protocol was scaled up for purification of a 5 liter E. coli culture, using a 10 ml Ni-agarose column, ˜23 mg of protein were obtained.

Purified trxA/tDXPS was treated with 10 units of thrombin/mg of protein for 30 minutes at room temperature to remove the thioredoxin portion of the fusion protein. However subsequent experiments showed that no difference in activity of the trxA/tDXPS chimera as compared to tDXPS.

### EXAMPLE 3

### LC/MS Analysis of DXPS Activity of trxA/tDXPS

Approximately 100 ng of the trxA/tDXPS protein prepared according to the method described in Example 2 was assayed overnight in 50 mM Tris, pH 7.5, 3 mM MgCl₂, 1 mM ThDp, 1 mM DTT, 1 mM pyruvate, 3 mM G-3-P, at 37° C. 100 μl of this reaction mix was reserved for determination of the pyruvate concentration as described in Example 4 below. The reaction was terminated in the remaining reaction mix by heating to 80° C. for two minutes. The mixture was then centrifuged @15,000×g to remove the protein. LC/MS analysis of the supernatant showed that in the presence of the trxA/tDXPS protein, DXP was produced while pyruvate and G-3-P were depleted.

### EXAMPLE 4

### Analysis of DXPS Activity By Determination of Pyruvate Concentration Using a Lactate Dehydrogenase Assay

We discovered that DXPS activity can be assessed by monitoring the disappearance of the substrate pyruvate. Pyruvate concentration can be determined indirectly by analysis of the conversion of pyruvate and NADH to lactic acid and NAD in the presence of lactate dehydrogenase. This second reaction can be monitored as a decrease in NADH concentration as the reaction proceeds. The concentration of NADH can be determined by measuring either the optical density of NADH at 340 nm or the relative fluorescence of NADH at 340 nm excitation/460 nm emission.

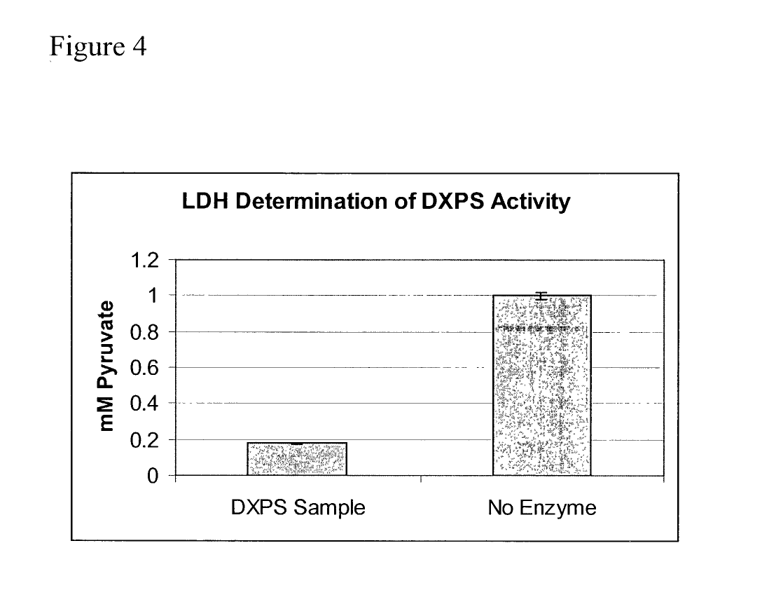

In the first assay, absorbance of NADH was measured. Briefly, 100 μl of reaction mix reserved in Example 3 were mixed with 100 μl of 50 mM Tris pH 7.5, 0.5 units/ml lactate dehydrogenase (LDH), 1 mM NADH, and incubated at room temperature for 10 minutes. Optical density of NADH was determined at 340 nm for the assay samples and a pyruvate standard curve. The pyruvate concentrations from the assay mixtures are shown in FIG. 4. Pyruvate was depleted from the reaction containing recombinant tDXPS. Values are the mean of triplicate determinations, standard deviation is indicated.

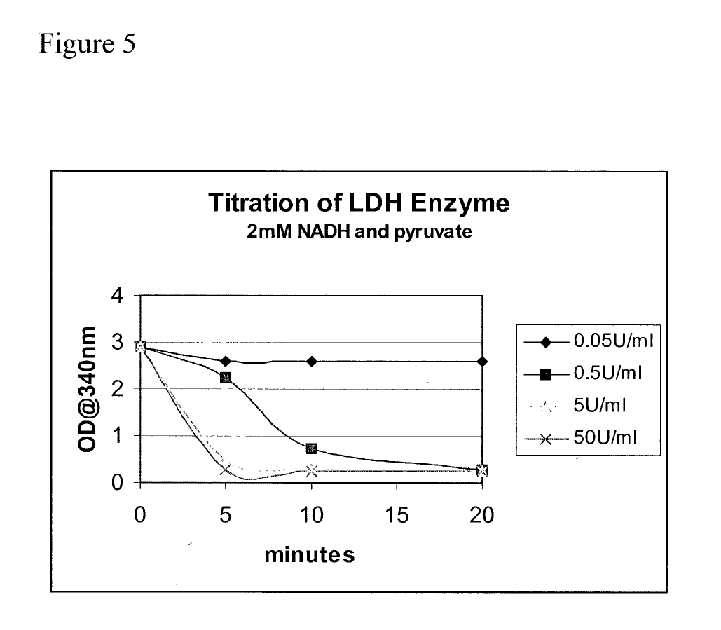

The optimal concentration of LDH concentration per pyruvate assay was determined as follows. 2 mM pyruvate and 2 mM NADH were mixed with an equal volume of LDH in Tris buffer. Absorbance at 340 nm was then determined at 0, 5, 10 and 20 minutes. The results are shown in FIG. 5. Values are the mean of duplicate determinations. Using 5 units/ml or more of LDH and adding in equal volumes to the reaction mix, the conversion of pyruvate to lactic acid and NAD is essentially completed in 5 minutes at room temperature at 2 mM NADH and pyruvate.

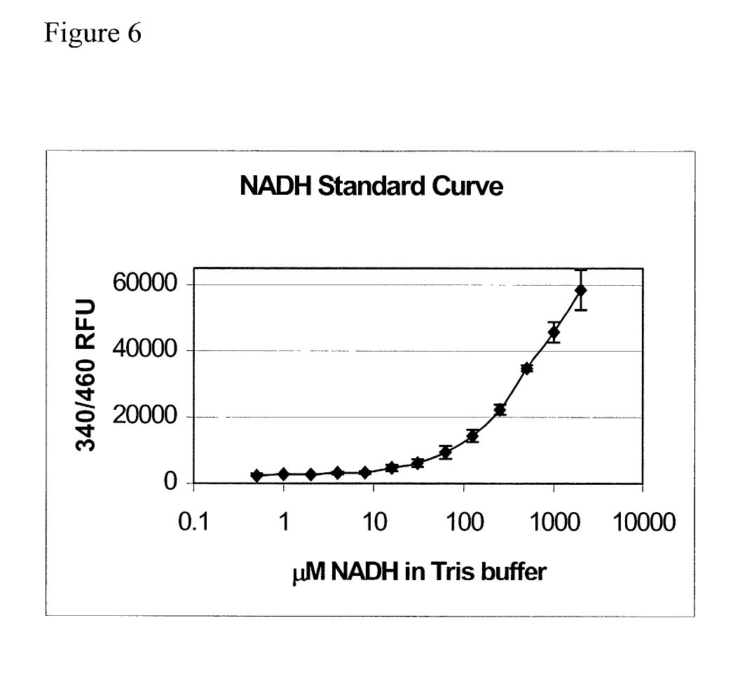

In the second assay, the concentration of NADH was determined by fluorescence. As a first step, a standard curve for NADH fluorescence was determined using 340 nm excitation/460 nm emission fluorescence. 50 μl/well of NADH solution was titrated in a 384 well plate and the relative fluorescence units (RFU) were determined. The automatic gain adjustment was used to set the gain level in the well with the highest concentration of NADH to give them a reading that was approximately 90% of the maximum value that the machine could determine. The results are shown in FIG. 6. Values are the mean of triplicate determinations. Standard deviation is shown as error.

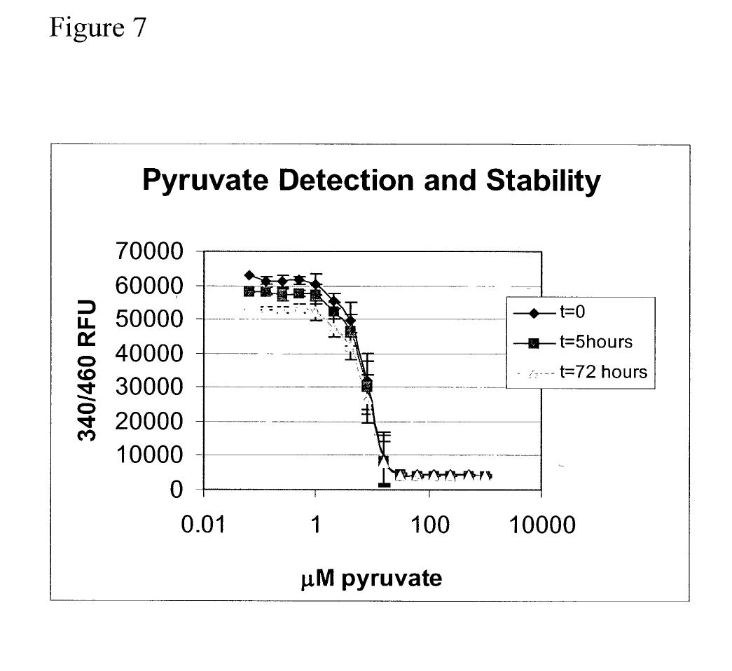

As a second step, a standard curve for pyruvate concentration was determined using detection of NADH fluorescence in the LDH assay. Detection buffer (50 mM Tris, pH 7.5, 5 units/ml of LDH, 25 μM NADH) was added to equal volumes of buffer containing various amounts of pyruvate. The relative fluorescence units (RFU) for NADH were determined at 340 nm excitation-460 nm emission in a solid white, Greiner 384 well plate. The detection buffer was made fresh for each time point, the pyruvate solution was added to the plate at 0 hours and the plate was incubated at room temperature until assayed for each time point. The results are shown in FIG. 7. Pyruvate shows good stability at room temperature and the detection of pyruvate concentration shows excellent repeatability. Values are the mean of triplicate determinations, standard deviation is indicated as error.

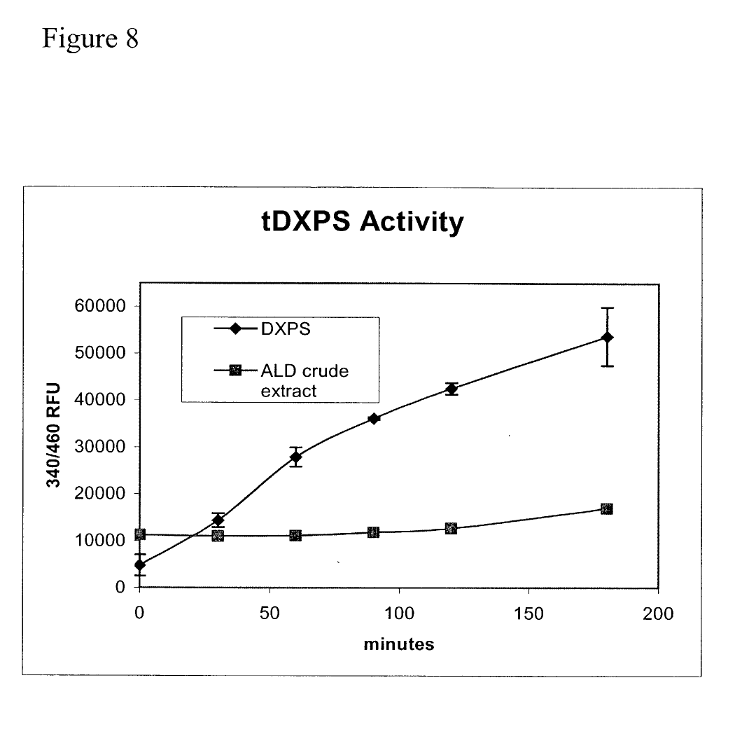

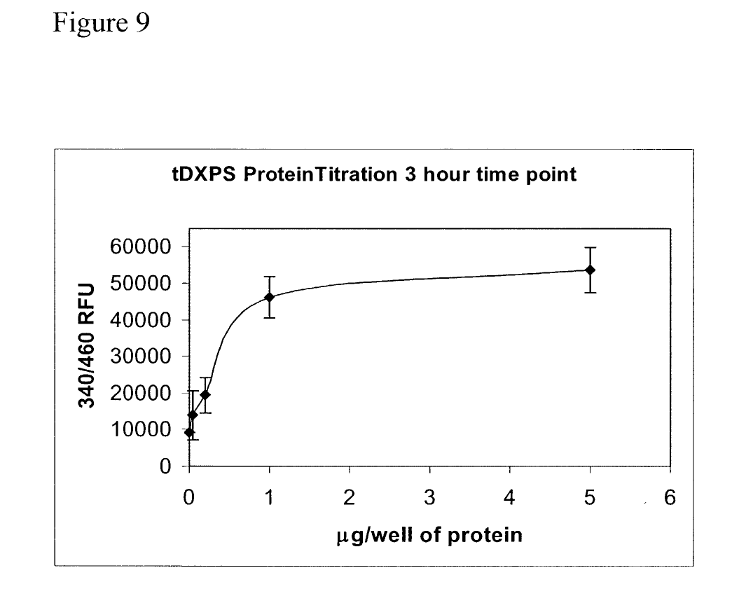

trxA/tDXPS activity was determined by fluorescence as follows. DXPS reactions were performed in a 384 well plate using 5 μg/well ofprotein, or crude E. coli supernate that does not contain the DNA for recombinant DXPS. The reaction mixture contained 50 mM Tris, pH 7.5, 50 μM glyceraldehyde 3-phosphate (G-3-P), 25 μM pyruvate, 25 mM DTT, 10 mM MgCl₂, and 1 mM thiamin diphosphate (ThDp). Reactions were performed at 37° C. in 50 μl, and were terminated with the addition of an equal volume of a 50 mM Tris (pH7.5) solution containing 5 units/ml lactate dehydrogenase and 25 μM NADH. The results are shown in FIG. 8. Values indicated for DXPS are the mean of triplicate determinations, with the error bars showing standard deviation, values for the crude supemate are single point determinations. This experiment was also done in a 96 well plate with similar results. Titration of tDXPS protein at a three hour time point showed that 1 ug/well of the purified tDXPS protein gave good activity in this assay (FIG. 9).

The thioredoxin/tDXPS fusion protein was cleaved with biotinylated thrombin at room temperature for 30 minutes, then assayed for DXPS activity. Activity was compared to an equal amount (1 μg/well) of unclipped protein (control). Removal of the thioredoxin portion of the fusion did not increase enzymatic activity of the protein as compared to protein that retained the thioredoxin tag.

### EXAMPLE 5

### Optimization of DXPS Assay

The concentrations of thioredoxin/tDXPS fusion protein, glyceraldehyde 3-phosphate, DTT, MgCl₂, ThDp, pyruvate, NADH and LDH were individually titrated in order to determine the optimal conditions for the assay of DXPS activity. The conditions and protocols chosen for high throughput screening of DXPS activity are as follows:

2×Assay Buffer

50 mM Tris, pH7.5

50 μM DL-glyceraldehyde 3-phosphate

60 μM pyruvate

50 μM NADH

5 mM DTT

2.5 mM MgCl₂

0.3 mM ThDp

Protein Dilution Buffer

50 mM Tris, pH7.5

5 mM DTT

2.5 mM MgCl₂

0.3 mM ThDp

Final Concentrations in the Assay

50 mM Tris pH 7.5

25 μM DL-glyceraldehyde 3-phosphate

30 μM pyruvate

25 μM NADH

5 mM DTT

2.5 mM MgCl₂

0.3 mM ThDp

Detection Buffer

50 mM Tris, pH 7.5

5 units/ml LDH

Assay Protocol

*All buffers need to be maintained @4° C. on the robot deck

1) Add 25 μl/well of 2×assay buffer by multidrop.

2) Add 5 μl/well of a test compound

3) Add 20 μl/well of protein (1 μg/well total protein) by multidrop

4) Incubate for three hours @37° C.

5) Add 50 μl/well of LDH detection buffer

6) Read fluorescence @340 em.-460 ex.

Assay Plate

Greiner solid white 384 well plate

Reagent List

DL-glyceraldehyde 3-phosphate Sigma Cat.#G 5251

Pyruvate, sodium salt Sigma Cat #P 2256

NADH, reduced form Sigma Cat #N 8129

LDH Sigma Cat #L 2500 One unit will reduce 1.0 μmol of pyruvate to lactate per nminute at pH 7.5 at 37° C.

ThDp (cocarboxylase) Sigma Cat #C 8754

MgCl₂ Sigma Cat #M 8266

DTT Sigma Cat #D 5545

384 well Statistical Analysis 50 μl of assay buffer plus 50 μl of detection buffer were added to a 384 well Greiner, solid white plate by multidrop. NADH concentration was then determined by fluorescence as described in the above protocol.

### EXAMPLE 6

### G-3-P is Not Necessary for the Assay of DXPS Activity

Even though a Km value could be determined for G-3-P, it was noted that there was still a significant difference between the no enzyme control and the no G-3-P control. To try and determine the reason for this, a DXPS reaction (1 mM pyruvate, +/−1 mM G-3-P, standard concentrations for the remaining assay components, 500 μl total volume, 10 μg tDXPS/reaction) was prepared and allowed to incubate overnight at 37° C. The reactions were terminated by heating the samples at 90° C. for two minutes. The samples were centrifuged at 15,000 rpm for 10 minutes to remove the precipitated protein, then analyzed by LC/MS. In the absence of enzyme, pyruvate could be detected. In the presence of enzyme and G-3-P, the appearance of DXP product was accompanied by the loss of pyruvate. However, in the presence of enzyme and absence of G-3-P, the pyruvate peak was lost without the concomitant formation of DXP. This shows the ability of DXPS to complete the first step in the DXP synthesis reaction in the absence of G-3-P.

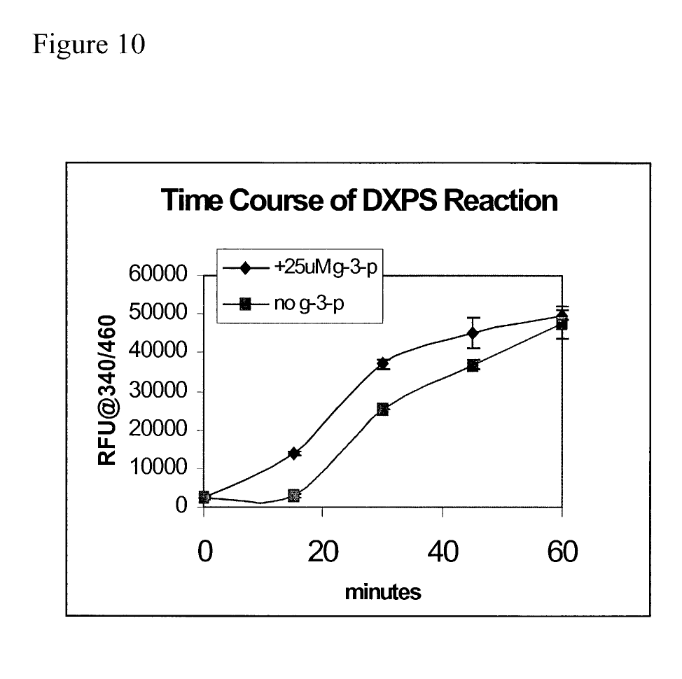

The rate of the DXPS reaction was compared in the presence and absence of G-3-P. 1 μg DXPS was mixed with 30 μM pyruvate, 50 mM Tris pH 7.5, 25 μM NADH, 5 mM DTT, 2.5 mM MgCl₂, 0.3 mM ThDp and in the presence or absence of 25 mM G-3-P. The reaction was incubated at 37° C. for 15, 30 or 60 minutes and then terminated by the addition of detection buffer (50 mM Tris, pH 7.5, 5 units/ml LDH). Fluorescence was measured at 340/460. The results are shown in FIG. 10. Again, even in the absence of G-3-P, the enzyme is capable of utilizing pyruvate as a substrate. Although the rate of the reaction is slower, the reaction can still go to completion. All values are in triplicate, standard deviation is indicated.

While the foregoing describes certain embodiments of the invention, it will be understood by those skilled in the art that variations and modifications may be made and still fall within the scope of the invention.

1. A polypeptide comprising SEQ ID NO:3.

2. An isolated polynucleotide comprising a nucleic acid encoding the polypeptide of SEQ ID NO:3.

3. An expression cassette comprising the polynucleotide of claim 3.

4. A method for identifying modulators of deoxyxylulose 5-phosphate synthase activity, comprising:
a) contacting pyruvate and optionally, glyceraldehyde 3-phosphate, with a deoxyxylulose 5-phosphate synthase, in the presence and the absence of at least one candidate modulator; and
b) comparing the concentration of pyruvate and/or glyceraldehyde 3-phosphate remaining after said contacting in the absence of said candidate modulator to said concentration in the presence of said candidate modulator.

5. The method of claim 4, wherein said optional glyceraldehyde 3-phosphate is omitted.

6. The method of claim 4, wherein said deoxyxylouse 5-phosphate is from a procaryote.

7. The method of claim 6, wherein said procaryote is selected from the group consisting of: Hemophilus influenzae, Rhodobacter capsulatus, Synechocystus sp. PCC6803, Bacillus subtilis, Helicobacter pylori and Mycoplasma tuberculosis.

8. The method of claim 4, wherein said deoxyxylulose 5-phosphate synthase is a plant deoxyxylulose 5-phosphate synthase.

9. The method of claim 4, wherein said deoxyxylulose 5-phosphate synthase is selected from the group consisting of: the polypeptide of SEQ ID NO:1 or an enzymatically active fragment thereof, the polypeptide of SEQ ID NO:2, and the polypeptide of SEQ ID NO:3.

10. The method of claim 4, wherein the concentration of pyruvate is determined.

11. The method of claim 10, wherein the concentration of pyruvate is determined by HPLC.

12. The method of claim 10, wherein the concentration of pyruvate is determined by contacting said pyruvate with lactate dehydrogenase and NADH and then determining the concentration of NADH.

13. The method of claim 12, wherein the concentration of NADH is determined by measuring the fluorescence of said NADH.

14. The method of claim 12, wherein the concentration of NADH is determined by measuring the absorbance of NADH.
